(19) United States
(12) Patent Application Publication
(10) Pub. No.: US 2022/0347422 A1
(43) Pub. Date: Nov. 3, 2022

(54) MEDICAL TUBE

(71) Applicant: NAN YA PLASTICS CORPORATION, Taipei (TW)

(72) Inventors: TE-CHAO LIAO, TAIPEI (TW); CHING-YAO YUAN, TAIPEI (TW); WEN-JUI CHENG, TAIPEI (TW); Min-Fan Chung, Taoyuan City (TW)

(21) Appl. No.: 17/535,657

(22) Filed: Nov. 25, 2021

(30) Foreign Application Priority Data
Apr. 30, 2021 (TW) 110115694

(51) Int. Cl. A61M 16/10; A61L 29/04

(52) U.S. Cl. CPC A61M 16/1095; A61M 16/1045; A61L 29/041; A61M 2202/02; A61M 2205/3368; A61M 2205/0216

(57) ABSTRACT

A medical tube is provided. The medical tube includes a hollow body and a gas conduit formed inside the hollow body for transporting gases. A material of the hollow body is a thermoplastic polyester elastomer. Based on a total weight of the thermoplastic polyester elastomer being 100 wt %, the thermoplastic polyester elastomer includes 50 wt % to 70 wt % of hard segments and 30 wt % to 50 wt % of soft segments.

### CROSS-REFERENCE TO RELATED PATENT APPLICATION

This application claims the benefit of priority to Taiwan Patent Application No. 110115694, filed on Apr. 30, 2021. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a medical tube, and more particularly to a medical tube that provides a gas to a patient and/or transports a gas away from the patient.

### BACKGROUND OF THE DISCLOSURE

A medical tube can be fluidly communicated with certain equipment and instruments to form a channel or a circulation loop, so as to provide a gas to a patient or transport a gas away from the patient. For example, the medical tube can be connected with a ventilator or an anesthesia machine.

In some cases, the ventilator is connected with a humidifier, so that the gas undergoes a heating treatment and a humidifying treatment before being provided to the human body through the medical tube. Due to the humidifier, internal organs of the patient can be prevented from dehydration, and a recovery time of the patient after a surgical operation can be shortened.

However, a common medical tube that is currently available on the market has a poor thermal insulating effect, so that a temperature of the treated gas decreases along with a length of the medical tube. When the temperature of the treated gas is decreased, a saturated humidity of the treated gas is also decreased. Accordingly, a condensate is easily formed on an inner wall of the medical tube during the transportation of the treated gas, so that a gas conduit is prone to blockage. In addition, the formation of the condensate may lead to bacterial growth.

In order to prevent the formation of the condensate, the length of the medical tube can be shortened, so as to reduce time needed for transporting the treated gas and decrease energy loss, or a thermal insulation sleeve can be mounted on the medical tube to decrease a cooling rate of the treated gas. However, whether through shortening the length of the medical tube or through mounting the thermal insulation sleeve, usage convenience of the medical tube will be decreased. Further, a flexibility of the medical tube will be negatively influenced by the mounting of the thermal insulation sleeve. Therefore, the medical tube on the market still has room for improvement.

### SUMMARY OF THE DISCLOSURE

In response to the above-referenced technical inadequacies, the present disclosure provides a medical tube.

In one aspect, the present disclosure provides a medical tube. The medical tube includes a hollow body and a gas conduit formed inside the hollow body for transporting gases. A material of the hollow body is a thermoplastic polyester elastomer. Based on a total weight of the thermoplastic polyester elastomer being 100 wt %, the thermoplastic polyester elastomer includes 50 wt % to 70 wt % of hard segments and 30 wt % to 50 wt % of soft segments.

In certain embodiments, the hard segments are formed from an aromatic polyester, and the soft segments are formed from an aliphatic polyester or an aliphatic polyether.

In certain embodiments, the aromatic polyester forming the hard segments is selected from the group consisting of: polybutylene terephthalate and polyethylene terephthalate.

In certain embodiments, the aliphatic polyether forming the soft segments is selected from the group consisting of: polytetramethylene ether glycol and polyethylene glycol.

In certain embodiments, the hollow body has an inner wall and an outer wall that are opposite to each other. The gas conduit is surrounded by the inner wall. An outer convex part is formed on the outer wall.

In certain embodiments, the outer convex part has a spiral shape along an axial direction of the hollow body.

In certain embodiments, the outer convex part and the hollow body are integrally formed.

In certain embodiments, the inner wall has a smooth surface without any convex part.

In certain embodiments, a wall thickness of the hollow body ranges from 100 μm to 600 μm, and a condensation amount per unit length of the medical tube in 24 hours is less than 2.000 g/cm.

In certain embodiments, the wall thickness of the hollow body ranges from 10 μm to 100 μm, and the condensation amount per unit length of the medical tube in 1 hour is less than 0.005 g/cm.

Therefore, in the medical tube provided by the present disclosure, by virtue of “the material of the hollow body being the thermoplastic polyester elastomer” and “based on the total weight of the thermoplastic polyester elastomer being 100 wt %, the thermoplastic polyester elastomer including 50 wt % to 70 wt % of the hard segments and 30 wt % to 50 wt % of the soft segments,” the medical tube has good air permeability and the condensation amount is reduced during use.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic view showing a medical tube of the present disclosure being used in cooperation with a ventilator, a humidifier, and a respirator;

FIG. 2 is a partial cross-sectional view of the medical tube according to a first embodiment of the present disclosure;

FIG. 3 is a partial cross-sectional view of the medical tube according to a second embodiment of the present disclosure;

FIG. 4 is a partial cross-sectional view of the medical tube according to a third embodiment of the present disclosure; and

FIG. 5 is a partial cross-sectional view of the medical tube according to a fourth embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

Referring to FIG. 1, a medical tube 1 of the present disclosure can be used in cooperation with a ventilator 7, a humidifier 8, and a respirator 9. The medical tube 1 is fluidly communicated with the ventilator 7, the humidifier 8, and the respirator 9, thereby forming a circulation loop.

In a practical operation, a dry gas with a specific composition is produced by the ventilator 7, and the dry gas is transported to the humidifier 8 via the medical tube 1. The humidifier 8 can heat the dry gas such that a temperature of the dry gas is closer to a body temperature, and can humidify the dry gas. After being treated by the humidifier 8, the dry gas can be transported to the respirator 9 via the medical tube 1 for a patient to breathe in. On the other hand, the gas exhaled from the patient can also be transported to the ventilator 7 via the medical tube 1, so as to remove the exhaled air from the patient.

The medical tube 1 of the present disclosure has good air permeability. When a saturated humidity is decreased due to a temperature drop of the gas inside the medical tube 1, the redundant water vapor can pass through the medical tube 1 and be diffused to an external environment, rather than forming into a condensate in the medical tube 1. Accordingly, the medical tube 1 of the present disclosure can improve a problem of formation of the condensate on an inner wall of a conventional medical tube due to a poor thermal insulating effect thereof.

Specifically, when a temperature decreases, the redundant water vapor in the medical tube 1 tends to adsorb onto the inner wall of the medical tube 1. Subsequently, the water vapor can pass through the medical tube 1 and be diffused to the external environment, so as to reduce the water vapor in the medical tube 1. In this way, the redundant water vapor will not completely form into the condensate in the medical tube 1.

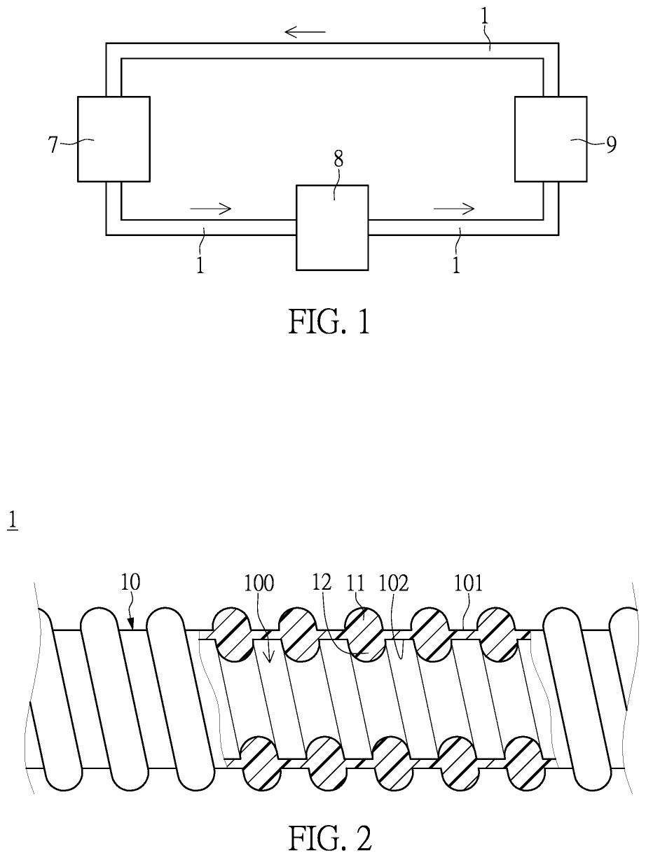

Referring to FIG. 2, FIG. 2 is a partial cross-sectional view of the medical tube 1 of the present disclosure. The medical tube 1 includes a hollow body 10 and a gas conduit 100 formed inside the hollow body 10 for transporting gases. The hollow body 10 has an outer wall 101 and an inner wall 102 that are opposite to each other. The gas conduit 100 is surrounded by the inner wall 102 of the hollow body 10.

The hollow body 10 is flexible to improve the convenience of use. The hollow body 10 can be transparent, semi-transparent, or opaque. Preferably, the hollow body 10 is transparent or semi-transparent, so that users can observe whether or not there is any pollutant or stagnant water in the gas conduit 100 to be cleaned. The gas conduit 100 is substantially a cylindrical channel, but is not limited thereto. The gas conduit 100 can be channels having other geometric shapes, such as a square channel or a triangular channel.

A material of the hollow body 10 is a thermoplastic polyester elastomer (TPEE). The thermoplastic polyester elastomer has a good processability, which can be applied in injection molding, blown film molding, or extrusion molding. In addition, the thermoplastic polyester elastomer upholds the softness and elasticity of rubber and the rigidity and chemical stability of engineering plastics.

The thermoplastic polyester elastomer of the present disclosure is a block copolymer which includes hard segments and soft segments arranged in alternation. The thermoplastic polyester elastomer of the present disclosure is synthesized from specific hard segments and soft segments, and a weight ratio of the hard segments to the soft segments is controlled.

Based on a total weight of the thermoplastic polyester elastomer being 100 wt %, the thermoplastic polyester elastomer includes 50 wt % to 70 wt % of the hard segments and 30 wt % to 50 wt % of the soft segments. Preferably, an amount of the hard segments is higher than an amount of the soft segments in the thermoplastic polyester elastomer. More preferably, the thermoplastic polyester elastomer includes 55 wt % to 65 wt % of the hard segments and 35 wt % to 45 wt % of the soft segments.

In the present disclosure, the hard segments of the thermoplastic polyester elastomer are formed from an aromatic polyester. The aromatic polyester can be copolymerized from an aromatic dicarboxylic acid and an aliphatic diol, or from an aromatic dicarboxylic acid and an alicyclic diol. The aromatic dicarboxylic acid can be, but is not limited to: benzenedicarboxylic acid, diphenyl dicarboxylic acid, or naphthalene dicarboxylic acid. The aliphatic diol can be, but is not limited to: ethylene glycol, propylene glycol, butylene glycol, or hexylene glycol. The alicyclic diol can be, but is not limited to: cyclohexanediol.

In an exemplary embodiment, the aromatic polyester to form the hard segments can be selected from the group consisting of: polyethylene terephthalate (PET) and polybutylene terephthalate (PBT), but is not limited thereto.

The soft segments of the thermoplastic polyester elastomer are formed from an aliphatic polyester or an aliphatic polyether. The aliphatic polyester can be copolymerized from an aliphatic dicarboxylic acid and an aliphatic diol, or be copolymerized from an aliphatic dicarboxylic acid and an alicyclic diol. The aliphatic polyether can be copolymerized from the aliphatic diol, or be ring-opening polymerized from an alkylene oxide and the aliphatic diol. The aliphatic dicarboxylic acid can be, but is not limited to: propionic acid, caproic acid, heptanoic acid, caprylic acid, or lauric acid. The aliphatic diol can be, but is not limited to: ethylene glycol, propylene glycol, butylene glycol, or hexylene glycol. The alicyclic diol can be, but is not limited to: cyclohexanediol. The alkylene oxide can be, but is not limited to: ethylene oxide, propylene oxide, or tetrahydrofuran. Specifically, a number average molecular weight of the soft segments ranges from 500 g/mol to 5000 g/mol. Preferably, the number average molecular weight of the soft segments ranges from 1000 g/mol to 4000 g/mol.

In an exemplary embodiment, the aliphatic polyether to form the soft segments is selected from the group consisting of: polyethylene glycol (PEG) and polytetramethylene ether glycol (PTMEG), but is not limited thereto.

In a process to synthesize the thermoplastic polyester elastomer, the aliphatic dicarboxylic acid and the aliphatic diol can be mixed and copolymerized, the aliphatic dicarboxylic acid and the alicyclic diol can be mixed and copolymerized, or the alkylene oxide and the aliphatic diol can be mixed and ring-opening polymerized, so as to form a precursor resin composition. Then, monomers required for synthesizing the hard segments, such as aromatic dicarboxylic acid, aliphatic diol, or alicyclic diol, are added into the precursor resin composition. After the synthesis, the thermoplastic polyester elastomer of the present disclosure can be obtained. The aforementioned steps are provided merely for illustrative purposes, and are not meant to limit the scope of the present disclosure.

### First Embodiment

Referring to FIG. 2, the medical tube 1 provided in a first embodiment of the present disclosure is a corrugated tube. An outer convex part 11 is formed on the outer wall 101 of the hollow body 10, and the outer convex part 11 and the hollow body 10 are integrally formed. An inner convex part 12 is formed on the inner wall 102 of the hollow body 10, and the inner convex part 12 and the hollow body 10 are integrally formed. Specifically, the outer convex part 11 and the inner convex part 12 are respectively formed on opposite surfaces of the hollow body 10. A location of the outer convex part 11 corresponds to a location of the inner convex part 12. The outer convex part 11 and the inner convex part 12 respectively have a spiral shape along an axial direction of the hollow body 10 by a same pitch.

A wall thickness of the hollow body 10 ranges from 50 μm to 600 μm, such that the medical tube 1 can have good air permeability to reduce the formation of the condensate. In addition, the medical tube 1 can conform to leakage limits specified in the ISO 5367:2000 standard.

In the first embodiment, the thermoplastic polyester elastomer of the present disclosure that includes the specific hard segments and soft segments is formed through copolymerization, and then the thermoplastic polyester elastomer is compressed into a sheet. Subsequently, the sheet is wound on a mold. After being heat-compressed, melted, and demolded, the medical tube 1 of the present disclosure can be obtained.

### Second Embodiment

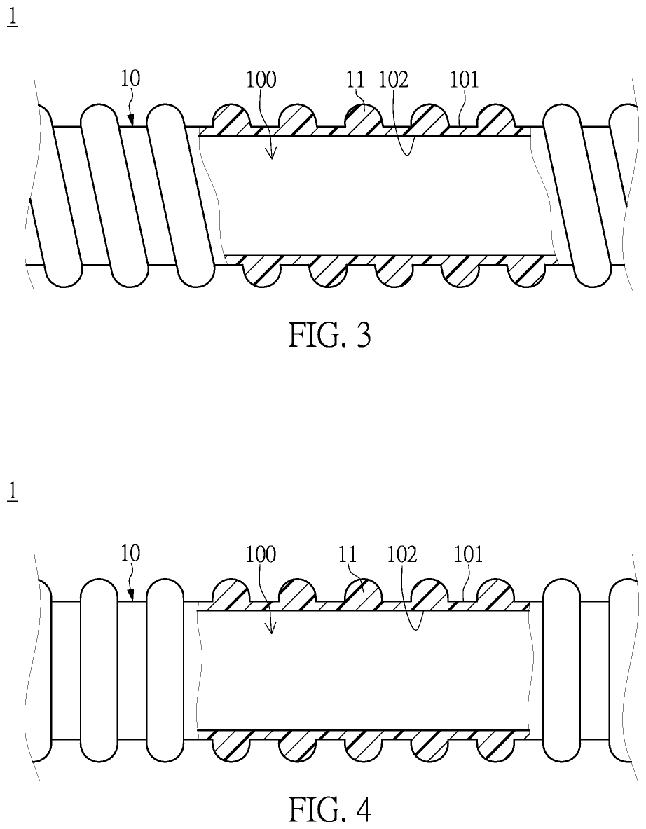

Referring to FIG. 3, the medical tube 1 provided in a second embodiment of the present disclosure is another corrugated tube. The hollow body 10 of the second embodiment is similar to the hollow body 10 of the first embodiment. The outer convex part 11 is formed on the outer wall 101 of the hollow body 10. The difference resides in that the inner wall 102 of the hollow body 10 of the second embodiment is a smooth surface without any convex part.

Due to the smooth surface, the water vapor is less likely to adsorb onto the inner wall 102 of the hollow body 10. Instead, the redundant water vapor is diffused to the outside of the hollow body 10. Therefore, the problem of the formation of the condensate on the inner wall 102 of the hollow body 10 due to the poor thermal insulating effect of the hollow body 10 can be improved.

### Third Embodiment

Referring to FIG. 4, the medical tube 1 provided in a third embodiment of the present disclosure is yet another corrugated tube. The hollow body 10 of the third embodiment is similar to the hollow body 10 of the second embodiment. The outer convex part 11 is formed on the outer wall 101 of the hollow body 10, and the inner wall 102 of the hollow body 10 is a smooth surface (without any convex part). The difference resides in that the outer convex part 11 of the third embodiment includes a plurality of ring parts that are formed on the outer wall 101 of the hollow body 10 in an equidistant and parallel manner.

### Fourth Embodiment

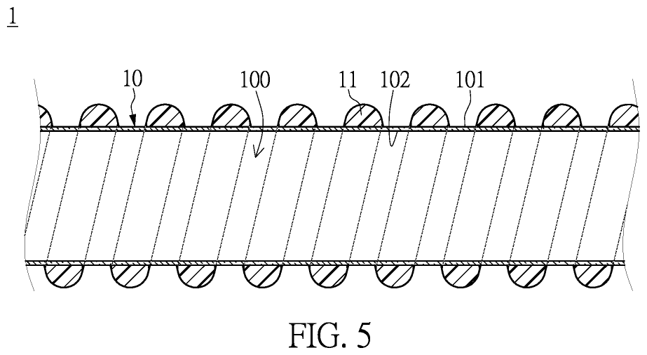

Referring to FIG. 5, the medical tube 1 provided in a fourth embodiment of the present disclosure is a spiral tube. The outer convex part 11 is spiraled on the outer wall 101 of the hollow body 10, and the outer convex part 11 is formed along the axial direction of the hollow body 10 by the same pitch. The inner wall 102 of the hollow body 10 is a smooth surface (without any convex part). In an exemplary embodiment, the outer convex part 11 and the hollow body 10 are integrally formed.

The wall thickness of the hollow body 10 ranges from 10 μm to 100 μm. In the fourth embodiment, the wall thickness of the hollow body 10 is thin. In order for the medical tube 1 to have sufficient mechanical strength, the outer convex part 11 is configured to support the hollow body 10 and maintain a shape of the hollow body 10.

In the fourth embodiment, the thermoplastic polyester elastomer of the present disclosure that includes the specific hard segments and soft segments is formed through copolymerization, and then the thermoplastic polyester elastomer is compressed into a sheet. Subsequently, the sheet is wound on a mold, and the outer convex part 11 is spiraled on the outer wall 101 of the hollow body 10 along the axial direction of the hollow body 10 by the same pitch, so as to support the hollow body 10 and maintain the shape of the hollow body 10.

### Experimental Data Test

To prove that the medical tube of the present disclosure has good air permeability and a condensation amount can be reduced, the medical tube of the second embodiment is connected with the ventilator and the humidifier to simulate the actual operation and compare experimental results.

The material of the medical tube in Examples 1 to 3 is the thermoplastic polyester elastomer, in which the hard segments are PBT and the soft segments are PEG A weight ratio of the hard segments to the soft segments is 60:40. The material of the medical tube in Comparative Example 1 is ethylene-vinyl acetate copolymer (EVA), which is the same as the material of a commercial medical tube. The materials of the medical tubes, the wall thickness of the medical tube, and the condensation amount per unit length in 24 hours in each of Examples 1 to 3 and Comparative Example 1 are listed in Table 1.

Parameters set in the ventilator include: an inflation frequency of 12 times per minute, an inflation volume of 600 ml, an inflation duration of 1 second, a deflation duration of 4 seconds, a deflation pressure of 5 cm H₂O, and an oxygen content of 21%. Parameters set in the humidifier include: a target temperature of 35.5° C. and an absolute humidity of 30 mg/L. A temperature of the external environment ranges from 23° C. to 26° C., and a relative humidity of the external environment ranges from 40% to 60%.

TABLE 1

| | | | Condensation |
|---|---|---|---|
| | | | amount per |
| | | Wall | unit length |
| | | thickness | in 24 hours |
| | Material | (μm) | (g/cm) |
| Example 1 | TPEE (hard | 300 | 0.985 |
| Example 2 | segments: soft | 400 | 1.234 |
| Example 3 | segments = 60:40) | 500 | 1.834 |
| Comparative | EVA | 400 | 4.368 |
| Example 1 | | | |

### Example 1

According to Table 1, the medical tube 1 of the present disclosure has good air permeability. When the wall thickness of the medical tube 1 ranges from 100 μm to 600 μm, the condensation amount per unit length in 24 hours is lower than 3.000 g/cm. Preferably, when the wall thickness of the medical tube 1 ranges from 100 μm to 600 μm, the condensation amount per unit length in 24 hours is lower than 2.000 g/cm. More preferably, when the wall thickness of the medical tube 1 ranges from 100 μm to 600 μm, the condensation amount per unit length in 24 hours is lower than 1.800 g/cm.

In addition, the medical tube provided in the fourth embodiment is fluidly communicated with a cup of warm water (70° C.), so as to simulate the actual operation and compare experimental results. The material of the medical tube 1 provided in the fourth embodiment is the thermoplastic polyester elastomer, in which the hard segments are PBT and the soft segments are PEG. The weight ratio of the hard segments to the soft segments is 60:40. The material of the medical tube of the Comparative Example 2 is a foamed polyester, which is the same as the material of another commercial medical tube. The materials of the medical tubes, the wall thickness of the medical tube, and the condensation amount per unit length in 1 hour in each of Example 4 and Comparative Example 2 are listed in Table 2.

TABLE 2

| | | | Condensation |
|---|---|---|---|
| | | | amount per |
| | | Wall | unit length |
| | | thickness | in 1 hour |
| | Material | (μm) | (g/cm) |
| Example 4 | TPEE (hard | 20 | 0.002 |
| | segments: soft | | |
| | segments = 60:40) | | |
| Comparative | Foamed | 20 | 0.006 |
| Example 2 | polyester | | |

According to Table 2, the medical tube 1 of the present disclosure has good air permeability. When the wall thickness of the medical tube 1 ranges from 10 μm to 100 μm, the condensation amount per unit length in 1 hour is lower than 0.005 g/cm. Preferably, when the wall thickness of the medical tube 1 ranges from 10 μm to 100 μm, the condensation amount per unit length in 1 hour is lower than 0.004 g/cm. More preferably, when the wall thickness of the medical tube 1 ranges from 10 μm to 100 μm, the condensation amount per unit length in 1 hour is lower than 0.003 g/cm.

Apart from having good air permeability and a reduced amount of the condensate, the medical tube of the present disclosure further conforms to the requirements designated for the medical tube, such as the leakage limit test (ISO 5367:2000), the tensile test (ISO 5367:2000), the humidity test (ISO 8185:2007), the compatibility test (ISO 5367:2000), and the impedance test (ISO 5367:2000).

### Beneficial Effects of the Embodiments

In conclusion, the medical tube provided by the present disclosure by virtue of “the material of the hollow body 10 being the thermoplastic polyester elastomer” and “based on the total weight of the thermoplastic polyester elastomer being 100 wt %, the thermoplastic polyester elastomer including 50 wt % to 70 wt % of the hard segments and 30 wt % to 50 wt % of the soft segments,” the air permeability of the medical tube 1 is enhanced, thereby reducing the condensation amount of the medical tube.

Further, in the medical tube provided by the present disclosure, by virtue of “the hard segments being formed from the aromatic polyester, and the soft segments being formed from the aliphatic polyester or the aliphatic polyether,” the medical tube 1 has good air permeability and the condensation amount is reduced during use.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A medical tube, comprising a hollow body and a gas conduit formed inside the hollow body for transporting gases; wherein a material of the hollow body is a thermoplastic polyester elastomer, and based on a total weight of the thermoplastic polyester elastomer being 100 wt %, the thermoplastic polyester elastomer includes 50 wt % to 70 wt % of hard segments and 30 wt % to 50 wt % of soft segments.

2. The medical tube according to claim 1, wherein the hard segments are formed from an aromatic polyester, and the soft segments are formed from an aliphatic polyester or an aliphatic polyether.

3. The medical tube according to claim 2, wherein the aromatic polyester forming the hard segments is selected from the group consisting of: polybutylene terephthalate and polyethylene terephthalate.

4. The medical tube according to claim 2, wherein the aliphatic polyether forming the soft segments is selected from the group consisting of: polytetramethylene ether glycol and polyethylene glycol.

5. The medical tube according to claim 1, wherein the hollow body has an inner wall and an outer wall that are opposite to each other, the gas conduit is surrounded by the inner wall, and an outer convex part is formed on the outer wall.

6. The medical tube according to claim 5, wherein the outer convex part has a spiral shape along an axial direction of the hollow body.

7. The medical tube according to claim 5, wherein the outer convex part and the hollow body are integrally formed.

8. The medical tube according to claim 5, wherein the inner wall has a smooth surface without any convex part.

9. The medical tube according to claim 1, wherein a wall thickness of the hollow body ranges from 100 μm to 600 μm, and a condensation amount per unit length of the medical tube in 24 hours is less than 2.000 g/cm.

10. The medical tube according to claim 1, wherein a wall thickness of the hollow body ranges from 10 μm to 100 μm, and a condensation amount per unit length of the medical tube in 1 hour is less than 0.005 g/cm.
